(19) United States
(12) Patent Application Publication
(10) Pub. No.: US 2009/0275955 A1
(43) Pub. Date: Nov. 5, 2009

(54) Mastoid Antral Ventilation Tube

(76) Inventor: Ahmet Kutluhan, Ankara (TR)

Correspondence Address: VENABLE, CAMPILLO, LOGAN & MEANEY, P.C., 1938 E. OSBORN RD, PHOENIX, AZ, 85016-7234, US

(21) Appl. No.: 11/720,719

(22) PCT Filed: Mar. 16, 2005
(86) PCT No.: PCT/TR2005/000011

§ 371 Date: Jun. 1, 2007

(30) Foreign Application Priority Data
Dec. 3, 2004 (TR) 2004/03298

(51) Int. Cl. A61F 11/00

(52) U.S. Cl. 606/109

(57) ABSTRACT

In its broadest form, this invention enables the ventilation and drainage of the middle ear cleft via mastoid antral route, without disrupting the integrity of the eardrum. For this purpose, this tube is placed between mastoid antrum and the external ear canal after mastoidotomy (antrotomy). The tube has two cannulas, which are placed one within the other. One of these is the external cannula and it comprises a head portion made of a circular tab, a neck portion in a vertical plane to the head portion, the bent section making a right angle of 90 degrees and a longer shaft portion. Other cannula is the internal cannula and it comprises a thin, narrow and syringe-adapted-tipped canal and the canal used for the aspiration and drainage of the rest of the lumen. The external tip of the inner cannula proceeds from the external ear canal and ends pre- or retroauricularly. Through the syringe-adapted-tip of the inner cannula, serum or drugs may be administered.

The invention relates to a mastoid antral ventilation tube that aerates and drains the middle ear by the route of mastoid antrum. On the contrary to the ventilation tubes placed on the eardrum, this tube is a ventilation and drainage tube placed between the mastoid antrum and the external ear canal.

The middle ear cleft comprises the air cells of the eustachian tube, middle ear and the mastoid. Embryologically, this cleft develops from the first branchial pouch, first eustachian tube, then the middle ear and finally the mastoid antrum and the air cells form. The aeration of the middle ear is almost complete at birth. However, the aeration of the mastoid cells continue until 9 years of age. These three aerated cavities that make up the middle ear cleft are connected to one another. Eustachian tube connects the middle ear to the nasopharynx. Rather small and narrow canals called Istmus anterior and Istmus posterior connect middle ear to the mastoid air cells. The mucosa events occurring in the middle ear are not limited only to the middle ear, they extend also to mastoid antrum and cells through aforesaid connections. Even though the function of the mastoid air cells is not known exactly, they are commonly accepted as the air reservoir for the middle ear. Hence, especially in children, mastoid air cells contribute to the ventilation of the middle ear in case of obstruction or dysfunction of eustachian tube, in order to prevent the formation of the negative pressure. However, prolonged dysfunction of eusthacian tube, increase of negative pressure in the middle ear and the obstruction of istmus anterior and posterior cause effusion to form in both middle ear and the mastoid cells. Initially, the medical treatment is applied to eliminate this effusion. In patients who are refractory to medical treatment, a ventilation tube (myringotomy tube, tympanostomy tube, grommet tube) is placed on the eardrum by means of a surgical operation called myringotomy (paracentesis), in order to ventilate the middle ear and drain the effusion. In this way, a cavity is provided to equate the pressure between the middle ear and the external medium and to permit the drainage of effusion. When the eardrum heals spontaneously afterwards, the tube is extruded, leaving a usually healthy eardrum. This operation has found a rather widespread use during the recent years. Although these tubes have provided important contributions to the treatment of the middle ear effusion, they have failed to provide the permanent and desired achievement in an non-neglectable number of patients. It was even reported that the long term outcomes in the patients with and without tube placed was similar. Moreover, many complications and sequelae to these tubes have been reported until present time. The object of the mastoidectomy, which is a surgical operation applied to open the mastoid antrum and air cells as the last resort for the patients who exhibit no improvement despite the repetitive applications of myringotomy tube, is to open the connections between the middle ear and the mastoid cells.

Myringotomy tubes have important disadvantages. As these tubes have a fairly small size, surgical skills and experience are required to place them on the eardrum. These tubes may frequently become obstructed, they are hard to aspire and they are not suitable for medical administration for the children. They do not provide information as to the potency of connection between the middle ear and the mastoid air cells. In connection with the myringotomy tube application, the complications and sequels such as chalk patches (myringosclerosis) on eardrum, perforation, discharge, retraction etc. may exist. The duration of these tubes is quite variable based on the reaction of the body, course of the middle ear disease and healing of the wound. Particularly in children, it is very difficult to open the myringotomy tube in case it becomes obscured during or immediately after the operation. As the air buffer in the middle ear is canceled by myringotomy tubes, the transport of mucus and bacteria from the nasopharynx to the middle ear through the eustachian tube is facilitated. Therefore, these tubes disable the functions of the eustachian tube during their period of stay in the patient. In addition, placement of these on the graft during the frequent practices of tympanoplasty brings about disadvantages both in wound healing and surgical intervention.

This invention aerates and drains the middle ear cleft by way of mastoid antrum. It re-established the connection between the middle ear and the mastoid air cells, and equates the pressure of both middle ear and the mastoid air cells with the external medium. The mucosal healing of the aerated cavities of the middle ear cleft is provided in a natural way, without the need for any intervention to the eardrum. A more permanent healing of disease will be provided, and unwanted complications and sequels are avoided. The mastoid antral ventilation tube can be used in children and the adult for all the diseases where the middle ear needs aeration and drainage.

The advantage of the mastoid antral ventilation tube over the myringotomy tubes placed on the eardrum is that it enables the eardrum to preserve its natural form, since no intervention to eardrum is involved. Via inner cannula, drug administration is possible to antrum, and therefore to the middle ear cleft. By this application, it is possible to control the patency of the passages from the mastoid antrum up to nasopharynx (mastoid antrum-middle ear and middle ear-nasopharynx) with sweet serums, which provides a very advantageous test for evaluation of the treatment. Since this tube has a rather larger inner diameter as compared to that of a myringotomy tube, the extent of mastoid and middle ear aeration will be greater, thereby it will contribute to a treatment concluding in a short time. In case of the middle ear aeration being considered to be insufficient in tympanoplasties, this tube may be easily applied. This tube covers all the indications where myringotomy tubes are used, and it may have wider field of use as compared to those.

The description of the figures which would help better understand the mastoid antral ventilation tube according to the invention:

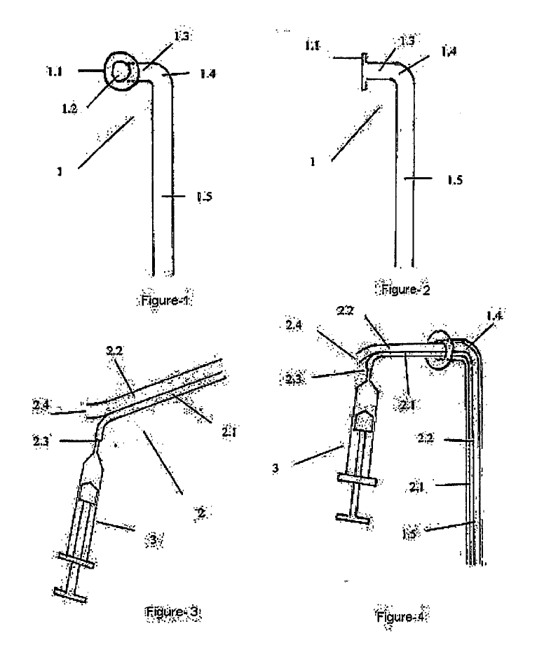

FIG. 1: Perspective view of the external cannula of mastoid antral ventilation tube

FIG. 2: Lateral view of the external cannula of mastoid antral ventilation tube

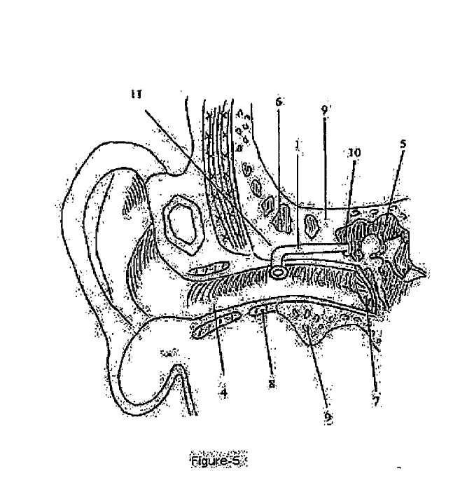

FIG. 3: Vertical cross-section showing the canals of the inner cannula of mastoid antral ventilation tube

FIG. 4: Perspective view of the inner and external cannula of mastoid antral ventilation tube together

FIG. 5: Perspective view of the external cannula of the mastoid antral ventilation tube placed in the ear

The description of the part numbers mentioned in the figures to help better understand the invention

1—External cannula of ventilation tube

1.1—Tab of external cannula

1.2—Lumen

1.3—Neck of external cannula

1.4—Bend of external cannula

1.5—Shaft of external cannula

2—Inner cannula of ventilation tube

2.1—Thin and narrow canal of inner cannula

2.2—Drainage canal of inner cannula

2.3—External tip of the thin and narrow canal of inner cannula

2.4—External tip of the drainage canal of the inner cannula

3—Syringe

4—External ear canal

5—Middle ear

6—Mastoid air cells

7—Eardrum

8—Cartilaginous of external ear canal

9—Bony portion of external ear canal

10—Mastoid antrum

The ventilation tube according to the invention designed for placement into mastoid antrum (10) comprises the following elements: Mastoid antral ventilation tube is composed of two main elements, external (1) and inner cannula (2). External cannula (1) comprises the sections of tab (1.1), neck (1.3), bend (1.4) and shaft (1.5). Lumen (1.2) of ventilation tube starts in the middle portion of the tab (1.1). The neck (1.3), bend (1.4) and shaft (1.5) sections of this cannula have tubular lumen. The first section of the tube after the tab (1.1) is the neck (1.3). The neck (1.3) is connected to the tab (1.1) at a certain angle.

After the neck (1.3), comes a bend (1.4) that forms an angle of preferably 80-90 degrees. The shaft (1.5) constitutes the final section of the external cannula (1). The length of the mastoid antral ventilation tube may be varied.

The inner cannula must have two canals. One of these canals must be a narrow and thin canal (2.1) placed on one side of the inner cannula. This canal is used for drug administration. The lumen of the inner cannula remaining from this canal constitutes the drainage canal (2.2). External tip (2.4) of this canal is used for aspiration. The two canals of the inner cannula (2) must end at the same level on mastoid antrum (10) without being separated from each other. Inner cannula (2) is longer than the external cannula (1), it leaves the external ear canal (4) and ends pre- or retroauricularly. The canals of the inner cannula (2) which remain outside the external ear canal (4) must be separated from one another and the external tip (2.3) of the thin-narrow canal must be terminated with a syringe adapter (3). Inner cannula (2) must be placed into the external cannula (1) neither loosely nor tightly, in such a way that it gets in and out easily. Canals (2.1, 2.2) of the inner cannula may be also in the form of completely independent tubes. Moreover, the inner cannula (2) may comprise a single tube. The inner and external cannulas (1, 2) that make up mastoid antral tube must be made of flexible and composite materials and they must not collapse.

In order to apply the mastoid antral ventilation tube being disclosed, which aerates and drains the middle ear (5) via mastoid antrum (10), a 1-2 cm postauricular skin incision is performed in consistency with the curvature of the auricle. Then mastoidotomy (antrotomy) is applied by a drill having a diameter of 2-3 mm from the location defined as Mc Evan triangle. Mastoid antrum is aspirated. Following the proper bleeding control, the mastoid antral ventilation tube along with its inner and external cannulas (1, 2) is placed by a mini-incision through the intersection of the external ear canal (4) with the bone (9) and the cartilage (8), and pushed into the site of mastoidotomy up to antrum (10). The tab (1.1) of the tube extends in the external ear canal (4) from the neck (1.3) to the site of mastoidotomy (11), and the shaft (1.5) extends up to the mastoid antrum (10). The shaft (1.5) length may be reduced on the condition that the tab (1.1) of the external cannula must remain in the external ear canal (4). The serum at body temperature is administered via the inner cannula (2) and aspirated. Inner cannula (2) is removed within few days following the disappearance of the aspirated mastoid effusion. External cannula (1) is maintained until the completion of the treatment. As the mastoid antral ventilation tube will not be spontaneously extruded, it is removed with the aid of a forceps.

As a sample application of the mastoid antral ventilation tube according to the invention, for a 5-year-old child, the tube must have an external cannula tab (1.1) of 3 mm, tube lumen (1.2) diameter of 1.5 mm, tube neck (1.3) of 2 mm and shaft (1.5) length of 1.5 cm. The inner cannula is longer than the external cannula, up to 10 cm.

The mastoid antral ventilation tube according to the invention is used in the treatment of the middle and inner ear diseases.

1. The invention relates to a mastoid antral ventilation tube, which is used in the treatment of the diseases related with the insufficiencies of aeration in middle ear, helps eliminate the inequilibrium of the inner ear fluids, enables the drug treatment for the diseases of middle and inner ear, makes it possible to aerate and drain the middle ear cleft via mastoid mitral path without disrupting the integrity of the eardrum, increases the success of the middle ear operations and is developed for placement into mastoid antrum, and it is characterized in that it comprises an external cannula (1) made up of the tab (1.1), neck (1.3), bend (1.4) and shaft (1.5) sections, and an inner cannula (2) made up of a thin and narrow canal (2.1) with a syringe-adapted-tip for drug administrations and the drainage canal (2.2) that provides the drainage-aspiration of mastoid antrum (10) with one end capable of extending up to mastoid antrum (10) via external cannula (1).

2. The ventilation tube according to claim 1 characterized in that lumen (1.2) of the ventilation tube starts at the middle portion of the tab (1.1), the projecting part of the external cannula.

3. The ventilation tube according to claim 1 characterized in that the neck (1.3), bend (1.4) and shaft (1.5) sections of the external cannula have tubular lumen.

4. The ventilation tube according to claim 1 characterized in that after the tab (1.1) of the external cannula, the first section of the tube is the neck (1.3), the neck (1.3) is connected to the tab (1.1) at a certain angle, after the neck (1.3), comes a bend (1.4) that forms a certain angle and the shaft (1.5) constitutes the final section of the external cannula (1).

5. The ventilation tube according to claim 1 characterized in that it is possible to vary the dimensions of the external cannula according to the patient whom it will be applied.

6. The ventilation tube according to claim 1 characterized in that the inner cannula has two canals, one of these canals is a narrow and thin canal (2.1) placed on one side of the inner cannula (2), the part of the inner cannula (2) remaining from the narrow and thin canal (2.1) constitutes the drainage canal (2.2).

7. The ventilation tube according to claim 1 characterized in that the outer edge (2.3) of the thin and narrow canal of the inner cannula (2) is suitable for drug administration, while external tip (2.4) of the drainage canal (2.2) is used for the purpose of drainage and/or aspiration.

8. The ventilation tube according to claim 1 characterized in that the two canals of the inner cannula (2) must end at the same level on mastoid antrum (10) without being separated from each other.

9. The ventilation tube according to claim 1 characterized in that inner cannula (2) is longer than the external cannula (1), it leaves the external ear canal (4) and ends pre- or retroauricularly.

10. The ventilation tube according to claim 1 characterized in that the canals of the inner cannula (2) which remain outside the external ear canal (4) must be separated from one another and the external tip (2.3) of the thin-narrow canal (2.1) must be terminated with a syringe adapter (3).

11. The ventilation tube according to claim 1 characterized in that inner cannula (2) must be placed into the external cannula (1) neither loosely nor tightly, in such a way that it gets in and out easily.

12. The ventilation tube according to claim 1 characterized in that canals (2.1, 2.2) of the inner cannula may be also in the form of completely independent tubes or alternatively, the inner cannula (2) may comprise a single tube.

13. The ventilation tube according to claim 1 characterized in that the inner and external cannulas (1,2) that make up mastoid antral tube must be made of flexible or composite materials and they must not collapse.

14. (canceled)

15. The ventilation tube according to claim 1 characterized in that it is possible to remove the external cannula (1) with the aid of a forceps, after the completion of the treatment.

16. The ventilation tube according to claim 1 characterized in that it is for single use.
